(19) United States
(12) Patent Application Publication
(10) Pub. No.: US 2011/0099693 A1
(43) Pub. Date: May 5, 2011

(54) WOMAN'S UNDERGARMENT

(76) Inventor: Karen Pratt, Carbondale, IL (US)

(21) Appl. No.: 12/613,271

(22) Filed: Nov. 5, 2009

(51) Int. Cl. A41B 9/04; A61F 13/15

(52) U.S. Cl. 2/406; 604/358

(57) ABSTRACT

A woman's undergarment (10) which hides the impression of a camel toe when worn beneath formfitting or tight fitting clothing. A panty has a pad (14) insertable into an enclosure (12) formed in the panty. The enclosure is sealed once the pad is fitted in it. The pad is shaped to substantially conform to the that of a woman's pubic area so to prevent an impression of the pubic area from showing through the panty and any clothing worn over the panty.

### CROSS REFERENCE TO RELATED APPLICATIONS

None

### STATEMENT REGARDING FEDERALLY SPONSORED RESEARCH OR DEVELOPMENT

N/A

### BACKGROUND OF THE INVENTION

This invention relates to women's underwear; and, more particularly, to a panty having a padded insert so to which prevents certain body contours from being visible when worn with tight fitting clothes.

It is not uncommon that when women wear form fitting or tight fitting clothes such as slacks, jeans, shorts, Capri or hot pants, or swimwear and the like for an imprint or impression of their labia majora to be visible. In slang terms, this is referred to as a “camel toe”, and can be a source of discomfort or embarrassment to the wearer. Previous approaches to addressing this problem have included an insert or cushion made of a smooth faced material which is inserted in a woman's panty, the insert presenting a smooth outer face which covers the camel toe. However, this approach has a number of problems. These include slippage or shifting of the insert during wear so that the insert does not always stay in place. The insert can also bunch up and therefore be uncomfortable to wear. The insert can even fall out of the pouch thereby negating the desired protection against camel toe.

### BRIEF SUMMARY OF THE INVENTION

The present invention is directed to a woman's undergarment or panty. A pouch is formed in the crotch portion of the panty and an insert is fitted in the pouch. The insert comprises a pad made of a foam or other padding material so for the pad to present a smooth outer surface. The shape of the insert generally corresponds to the contour of a woman's crotch. The length of the insert is such that when the panty is worn, the pad substantially covers the woman's pubic area so to prevent the appearance of a camel toe even when the woman wears form fitting or tight fitting clothes. The insert is enclosed in a wickable, anti-bacterial lining. The insert is sewn or otherwise secured in place so that it does not move or shift as wearer stands, sits, walks, or engages in other activities.

Other objects and features will be in part apparent and in part pointed out hereinafter.

### BRIEF DESCRIPTION OF THE SEVERAL VIEWS OF THE DRAWINGS

The objects of the invention are achieved as set forth in the illustrative embodiments shown in the drawings which form a part of the specification.

FIG. 1 is a simplified representation of a female “camel toe” condition;

FIG. 2 is perspective view of a woman's panty with an insert of the present invention; and,

FIG. 3 is bottom plan view of the panty.

Corresponding reference characters indicate corresponding parts throughout the several views of the drawings.

### DETAILED DESCRIPTION OF INVENTION

The following detailed description illustrates the invention by way of example and not by way of limitation. This description clearly enables one skilled in the art to make and use the invention, and describes several embodiments, adaptations, variations, alternatives and uses of the invention, including what is presently believed to be the best mode of carrying out the invention. Additionally, it is to be understood that the invention is not limited in its application to the details of construction and the arrangement of components set forth in the following description or illustrated in the drawings. The invention is capable of other embodiments and of being practiced or carried out in various ways. Also, it will be understood that the phraseology and terminology used herein is for the purpose of description and should not be regarded as limiting.

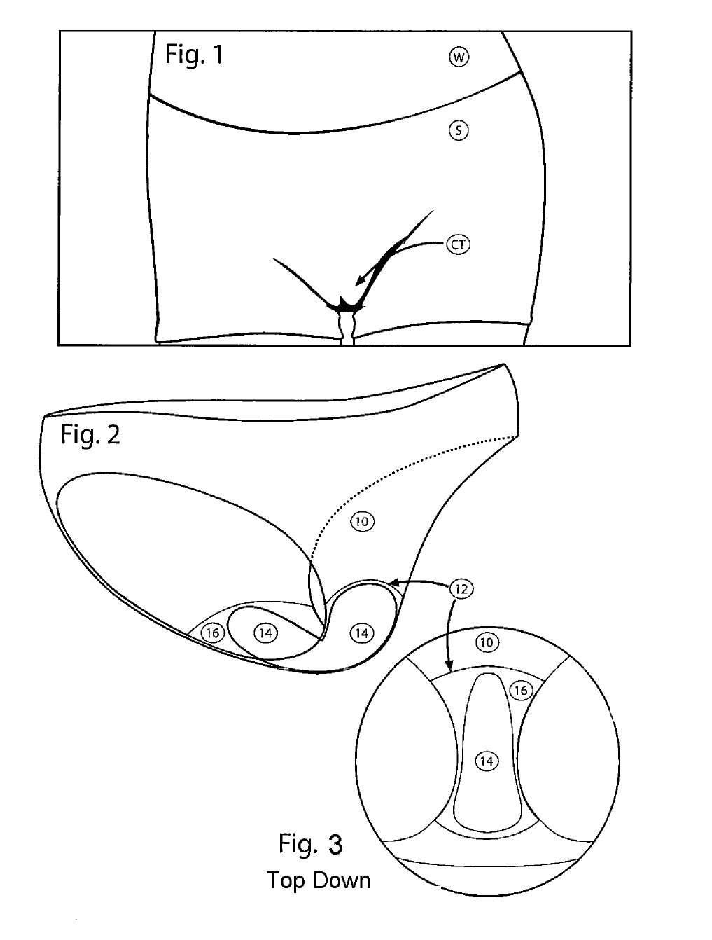

Referring to FIG. 1, a “camel toe” CT typically results when a woman W wears a tight fitting garment such as shorts S. In accordance with present invention, a panty indicated generally 10 in FIGS. 2 and 3, is designed to eliminate the appearance of this condition.

As shown in FIGS. 2 and 3, panty 10 is formed with a pouch or enclosure 12 which extends from the front of the panty, through the crotch area, to the rear of the panty. An insert or pad 14 which is generally shaped to the contour of the pubic and crotch areas of a woman's body is made of a relatively thin, flexible foam material. For example, the insert or pad is made of a polylaminate foam material or the like and has a thickness, for example, of ⅛″ (0.32 cm). The thinness of the insert makes the panty comfortable to wear. The material is also a perforate material which allows it to breathe.

Importantly, the outer surface of the pad is smooth. This is so that when the panty is worn beneath a form fitting or tight fitting garment, the insert covers the woman's pubic area and only a smooth appearance is presented to those looking at her. No imprint or impression of a camel toe will now appear when the panty is worn.

Next, pad 14 is covered or encased or enclosed in an anti-bacterial material 16. This material is also preferably a wicking material. When the panty is made, the pad and liner are inserted into pouch 12 with the open end of the pouch then sewn up or otherwise sealed so that pad 14 remains in place when the panty is worn.

Finally, those skilled in the art will appreciate that the insert and liner can be assembled into a variety of women's undergarments including different styles of a panty without departing from the scope of the invention.

In view of the above, it will be seen that the several objects and advantages of the present disclosure have been achieved and other advantageous results have been obtained.

Having thus described the invention, what is claimed and desired to be secured by Letters Patent is:

1. A woman's undergarment which hides the impression of a camel toe when worn beneath formfitting or tight fitting clothing comprising:
a panty; and,
a pad insertable in an enclosure formed in the panty, the enclosure being sealed once the pad is inserted therein, and the pad being shaped to substantially conform to the that of a woman's pubic area so to prevent an impression of the pubic area from showing through the panty and any clothing worn over the panty.

2. The undergarment of claim 1 in which the pad is made of a foam material having a smooth outer surface so clothing under which the garment is worn presents a smooth outer appearance.

3. The undergarment of claim 2 further including an anti-bacterial material covering the foam material from which the pad is made.

4. The undergarment of claim 3 in which the anti-bacterial material is further a wicking material.
